(19) United States
(12) Patent Application Publication
(10) Pub. No.: US 2022/0183661 A1
(43) Pub. Date: Jun. 16, 2022

(54) IMPLANTABLE ULTRASOUND GENERATING DEVICE FOR IMPLANTATION WITHIN A VERTEBRAL COLUMN

(71) Applicants: SORBONNE UNIVERSITE, PARIS (FR); ASSISTANCE PUBLIQUE - HÔPITAUX DE PARIS, PARIS (FR)

(72) Inventor: ALEXANDRE CARPENTIER, PARIS (FR)

(21) Appl. No.: 17/598,327

(22) PCT Filed: Mar. 27, 2020
(86) PCT No.: PCT/EP2020/058833

§ 371 Date: Sep. 27, 2021

(30) Foreign Application Priority Data
Mar. 28, 2019 (EP) 19305406.1

(51) Int. Cl. A61B 8/08; A61M 37/00

(52) U.S. Cl. CPC A61B 8/481; A61M 2210/1003; A61M 37/0092

(57) ABSTRACT

The present invention relates to an implantable ultrasound (US) generating device for implantation within the vertebral column of a vertebrate subject, wherein the implantable US generating device comprises at least one US generating transducer suitable for emitting US beam(s) with an oblique orientation with respect to a longitudinal axis of a vertebral column.

### TECHNICAL FIELD

The present invention relates to an implantable ultrasound generating device suitable to be implanted within the vertebral column of a vertebrate subject and dedicated to the treatment of spinal cord disorders. The device of the invention is particularly suited for transiently disrupting the blood-spinal cord barrier of a vertebrate subject, especially of a human.

### BACKGROUND ART

The spinal cord may be subject to various physiological disorders which induce different forms of pathologies, e.g. spinal cord tumor, spinal degenerative pathology, spinal cord inflammation, etc. Some available treatments include action of drugs on the spinal cord, which are generally administrated by intravenous route. However, the blood-spinal cord barrier (hereinafter “BSCB”) limits or prevents the penetration of therapeutic drugs in the spinal cord or nerve tissues. To circumvent this phenomenon, it is known to use spinal drug delivery catheters inserted in the spinal canal so that the drugs are directly located around the spinal cord. However, this strategy requires the use of fluid to be injected. Furthermore, the fluid generally penetrates to a limited and insufficient extent into spinal cord or spinal nerve tissues.

Recently, the inventors have proposed a method capable of causing the transient disruption of the blood-spinal cord barrier of a vertebrate subject, by concomitant pulsed ultrasound emission and intravenous contrast agent injection. This method leads to safe opening of the BSCB. However, aleatoric toxicity problems were observed, that may damage the targeted tissues.

To understand what was going on, the inventors analyzed the ultrasound acoustic field by in silico treatment simulations (FIG. 1). The nominal treatment pressure amplitude delivered during treatments is defined as the pressure that would have been delivered in non-attenuative free-field conditions (water). Simulation showed that, the presence of distal bone in the acoustic field causes ultrasound reflections and standing waves. Standing waves are the resultant of superposition of emitted waves with reflected waves by the surrounding bone of the spinal canal. Due to the close proximity of vertebral body anterior to the spinal cord, ultrasound reflection happens, amplifying the signal intensity, leading to unexpected pic acoustic pressure leading to intra medullary hematoma and then safety considerations.

Simulations for other vertebras (C6, T4, T9, L2) show similar pressure field patterns. Overall, 34±6% of the spinal canal ROI volume (mean±standard deviation for the 4 evaluated vertebras) received an acoustic pressure of 0-0.5× the nominal pressure, 47±5% between 0.5-1×, 17±3% between 1-1.5×, and 2%±1% above 1.5×. For comparison, if ultrasound reflection and attenuation are neglected (free-field condition), 48% of the spinal-canal ROI volume would have a pressure between 0-0.5× the nominal pressure and 52% of the ROI would be exposed with a pressure between 0.5-1× the nominal pressure.

### SUMMARY

To solve this problem, the inventors have developed an ultrasound (US) generating device suitable for implantation within the vertebral column of a vertebrate subject, which emits ultrasounds obliquely in the spinal canal, instead of radially (i.e., orthogonal to the longitudinal axis of the spinal canal). Such angulation allows for a significant reduction of standing waves since the ultrasound beam is reflecting obliquely on the bone, avoiding the incidence beam. This permits to get rid of the pic acoustic pressure in the spinal cord, avoiding over treatment areas and thereby bleeding side effects. The more the angulation is, the more hot point amplitude decreases. Advantageously, the angulation is comprised between 45° and 65°, with respect to a longitudinal axis of a vertebral column. Alternatively speaking, the angulation is comprised between 25° and 45°, with respect to an orthogonal axis of a vertebral column.

It is thus an object of the present invention to provide an implantable ultrasound (US) generating device for implantation within the vertebral column of a vertebrate subject, wherein the implantable US generating device comprises at least one US generating transducer suitable for emitting US beam(s) with an oblique orientation with respect to a longitudinal axis of a vertebral column.

In a particular embodiment, the US generating transducer is a plane US transducer with an oblique orientation within the implantable device. Alternatively, the US generating transducer is a plane US transducer with a longitudinal orientation within the implantable device, and the implantable device further comprises at least one lens to deviate US beam(s) in an oblique direction with respect to the longitudinal axis of the vertebral column.

### BRIEF DESCRIPTION OF THE FIGURES

FIG. 1 shows the simulated acoustic pressure field for an ultrasound exposure of a rabbit T4 vertebra after laminectomy through a 2-cm thick acoustically transparent gelatin pad.

FIG. 2 shows simulated acoustic pressure field (color overlay) in the T4 vertebra of a 1 kg rabbit (grayscale background: CT scan). A) sagittal profile of the acoustic pressure simulated when mimicking experimental conditions (accounting for gel coupling pad, laminectomy, attenuation in soft tissues, reflection from bone interfaces) using acoustic properties and geometry of bone determined from the CT data; B) corresponding 2D transverse profile. C) 1D Axial profiles of the acoustic pressure relative to free field conditions (water).

FIG. 3 illustrates an embodiment of an implantable US generating device, wherein the implantable device as a general shape of a vertebral arch and replace said posterior part of a vertebra. The implantable US generating device comprises plane US transducer with an oblique orientation within the implantable device.

FIG. 4 shows the implantable US generating device according to FIG. 3 within the vertebral column of a subject and the orientation of US beam when impacting the spinal cord of the subject.

FIG. 5 illustrates another embodiment of an implantable US generating device within the vertebral column of a subject and the orientation of US beam when impacting the spinal cord of the subject, wherein the plane US transducer has a longitudinal orientation, and the implantable device further comprises an oblique lens.

FIG. 6 illustrates another embodiment of an implantable US generating device within the vertebral column of a subject and the orientation of US beam when impacting the spinal cord of the subject, wherein the plane US transducer has a longitudinal orientation, and the implantable device further comprises two oblique lenses, with two opposite oblique surfaces.

FIG. 7 shows simulated acoustic pressure field (color overlay) for three different angulations, 0°, 30° and 45° with respect to the orthogonal axis, corresponding to 90°, 60° and 45° with respect to the longitudinal axis of a vertebral column.

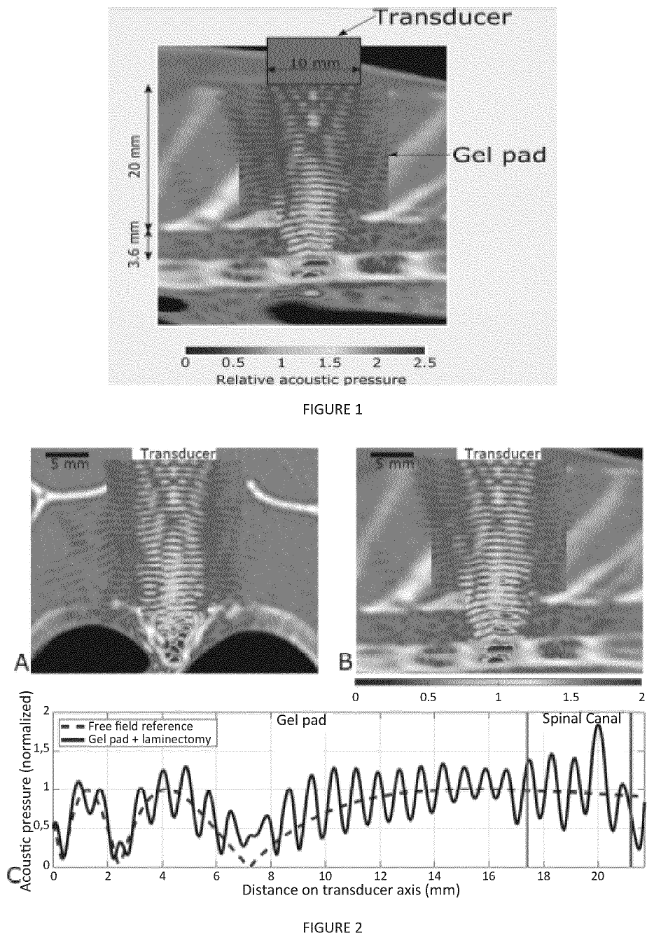

### DETAILED DESCRIPTION

The present disclosure will be best understood by reference to the following definitions.

### Definitions

In the context of the invention, the term “disrupting the BSCB”, “opening the BSCB” or “increasing the permeability of the BSCB” refer to an increased susceptibility of the BSCB to the passage of molecules therethrough that occurs without detectable damages of the spinal cord or spine.

The term “ultrasound beam”, “ultrasound wave” and “ultrasound” are used for designating sound waves with frequencies higher than 20 kHz. The ultrasound energy may be focused ultrasound or unfocused ultrasound to treat a large zone of the BSCB.

As used herein, “subject” refers to any vertebrate subject, especially a mammal, and in particular a “human”, i.e., a person of the species Homo sapiens, including man, woman, child and human at the prenatal stage. In one embodiment, a subject may be a “patient” who is awaiting the receipt of, or is receiving medical care or was/is/will be the object of a medical procedure, or is monitored for the diagnosis or the development of a disease.

Throughout the disclosure, various aspects of the invention can be presented in a range format. It should be understood that the description in range format is for convenience and brevity and should not be constructed as an inflexible limitation on the scope of the invention. Accordingly, the description of a range should be considered to have specifically disclosed all the possible subranges as well as individual numerical values within that range, range values being included.

Implantable Device

It is an object of the present invention to provide an implantable ultrasound (US) generating device for implantation within the vertebral column of a vertebrate subject, wherein the implantable US generating device comprises at least one US generating transducer suitable for emitting US beam(s) with an oblique orientation with respect to a longitudinal axis of a vertebral column

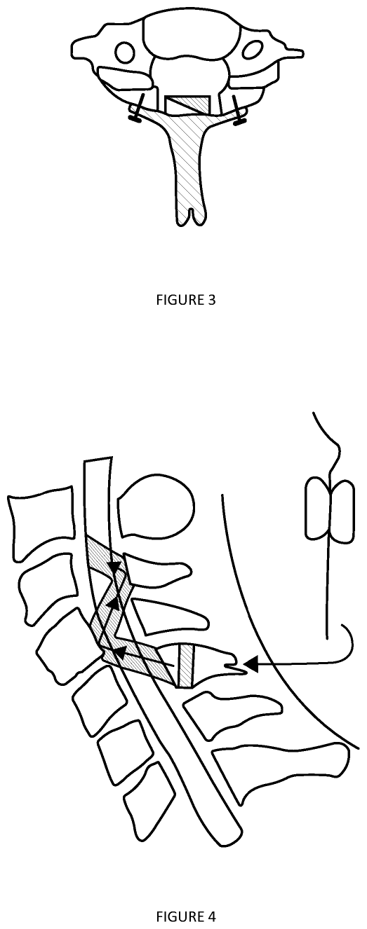

In the context of the invention, the expression “for implantation within the vertebral column” means that the implantable device is able to be inserted in the backbone of a subject, so that the US generating transducer would discharge within the spinal canal and the US beams would impact the spinal cord. In a particular embodiment, the implantable device as a general shape of the posterior part of a vertebra, i.e., the vertebral arch. Thus, the US generating device may replace partially a vertebra in the subject (see FIG. 3). Alternatively, the US generating device may be inserted within a vertebra, and preferably within the posterior part of a vertebra.

According to the invention, the implantable device may replace or be inserted within any one of the vertebrae of a subject. Particularly, if the subject is a human, the implantable device may replace or be inserted within at least one of the 33 vertebrae. Preferably, the implantable device may replace or be inserted within at least one of the vertebrae of the cervical spine (C1-C7) to address cervical spinal cord, thoraco-lombar spine (T7-L1) to address lombar spinal cord.

According to the invention, the US generating transducer is positioned within a body of the implantable device so that the US beams are emitted with an oblique orientation with respect to the longitudinal axis of the vertebral column in which said device is implanted. Advantageously, US beam(s) will be emitted with an angle between 45° and 65° with respect to the longitudinal axis of a vertebral column

FIG. 4 illustrates an embodiment of the present invention, wherein a US generating device replaces the posterior part of a vertebra. According to this embodiment, the US generating transducer is plane and disposed obliquely within the body of the implantable device, with respect to a longitudinal axis of said body. The US beams are emitted radially with respect to the longitudinal axis of the transducer, but because said transducer is inclined with respect to the longitudinal axis of the vertebral column, the US beams will also impact the spinal cord obliquely.

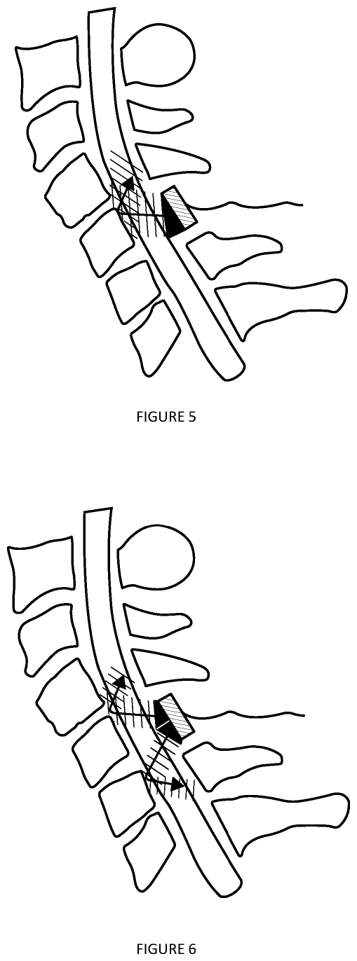

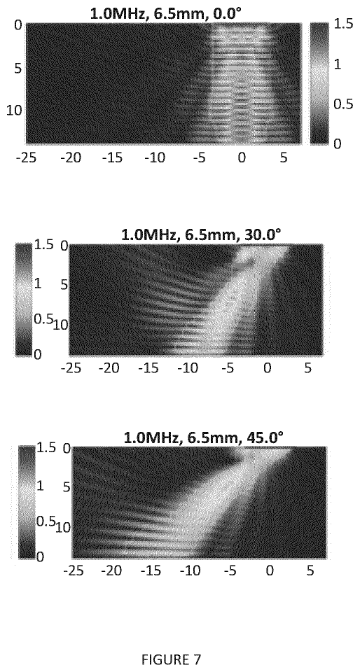

In another embodiment, US generating transducer is a plane US transducer with a longitudinal orientation within the implantable device, and the implantable device further comprises at least one lens to deviate US beam(s) in an oblique direction with respect to both the longitudinal axis of the vertebral column and longitudinal axis of the implantable device (see FIG. 5, wherein the device comprises one oblique lens and FIG. 6, wherein the device comprises two oblique lenses with opposite inclinations).

In a particular embodiment, the implantable device comprises two or more US generating transducers suitable for emitting US beam(s) with an oblique orientation with respect to a longitudinal axis of a vertebral column. Preferably, such a device comprises two US generating transducers. Advantageously, a first US generating transducer is suitable for emitting US beam(s) upward and a second US generating transducer is suitable for emitting US beam(s) downward with respect to the orientation of the vertebral column.

In case of several US generating transducers in an implantable device, the US generating transducers can be activated preferably sequentially. In another embodiment, the US generating transducers can be activated simultaneously and/or both sequentially and simultaneously, with the aim to avoid interferences between the beams.

In a particular embodiment, at least one US generating transducer comprises an array of several transducers. The transducers in the array have reduced dimensions as compared to a single transducer. Advantageously, they are uniformly disposed on a support bearing the array of transducers. This embodiment may be useful for adapting the emission frequencies and/or emission stages and/or beam steering and/or oblique emission within the spinal cord of the subject.

In a particular embodiment, the implantable device comprises transdermal connection means to be connected to an external electric supplier. To this end, the device can comprise a connection chamber to receive the transdermal electric connection. An internal or external electric supplier (i.e. battery) can be used for connection to the implantable device that can be remotely controlled for treatment activation.

If several implantable devices are implanted within a vertebral column (e.g., to cover a larger spinal cord area), it is possible to use a master implantable device which comprises the transdermal connection means, and the other implantable devices are deprived of transdermal connection means and are connected to the master implantable device. In such embodiment, all the implantable devices will emit US beams simultaneously. Alternatively, at least two implantable devices can comprise transdermal connection means. Advantageously, in case of several implanted devices at different level of the spinal cord, contiguous devices may be activated in desynchrony (i.e., sequentially) to avoid ultrasound wave superpositions/interferences between transducers. The external or internal generator will be able to manage such sequencing.

In another embodiment, US beams can be emitted by a same transducer with a frequency modulation, in order to reduce standing waves.

In another embodiment, US beams can be emitted by a phased array transducer in order to acquire a spinal cord echographic image, in order to emit obliquely by modifying phases and frequencies, in order to permit an electronic beam steering, in order to focalized ultrasound emission.

Ultrasound Beams

The purpose of the present invention is to provide an implantable device suitable for emitting US beams on the spinal cord of a subject in need thereof, in order to disrupt the BRSC. Alternatively or in addition, the implantable device of the invention may be used for generating slight hyperthermia to stimulate local immunity and/or to stimulate neuronal regeneration.

Advantageously, the US generating transducer of the implantable device is able to emit unfocused beams. Indeed, the inventors have shown that unfocused beam has a natural hot spot focal at a certain distant from the transducer. This distance d can be calculated by the following formula:

L=2√dλ

where L is the dimension of the ultrasound opening (i.e. diameter), and λ is the wave length (sound speed/frequency). As an example, for a 10 mm diameter unfocused plane transducer, emitting at 1 MHz, the natural focal hot spot is at 15 mm from the transducer. If one has to gain in safety for ultrasound emission within the spinal cord, avoiding standing waves, it is necessary to avoid that the hot spot happens at the place of bone reflection (on the bone surface). Ideally the hot spot should be chosen to be in the middle of the spinal cord. Then the distance of the transducer from the spinal cord has to be adapted to match this requirement.

Advantageously, frequency of ultrasound emission is chosen between 0.3 and 3 MHz. By reducing frequency (ie. to about 0.5 MHz), not only bone reflection can be decreased on axial emissions, but the beam will be less focalized leading to a larger treatment zone. Then the acoustic pressure is more dispersed so that less intensity reaches the bone inducing again less reflection intensity.

According to the invention, the unfocused US beams can be applied to the spinal cord of the subject, with a pressure level ranging from 0.3 to 2 MPa. In the context of the invention, the “pressure level” refers to the maximum acoustic pressure measured in the acoustic field of the emitter in water. Advantageously, the unfocused US beams are applied within a pressure range of 0.5 MPa to 1.25 MPa, preferably within a pressure range of 0.8 MPa to 1 MPa.

In the context of the invention, the value of the pressure level corresponds to the value of the pressure level coming out of the emitter. The pressure onto the spinal cord may be lower, because of the potential attenuation of intervening tissues. Generally speaking, the attenuation may be at most of 30%. It is a purpose of the present invention to reduce this phenomenon by introducing bone replacement. On the other hand, the pressure onto the spinal cord may be higher, because of the potential bone reflection. Generally speaking, the amplification may be at most of 30%. It is a purpose of the present invention to reduce this phenomenon by introducing an oblique emission.

Treatment of a Spinal Cord Disorder

The implantable device of the present invention is particularly suited to be used in treating a spinal cord disorder by transiently disrupting the blood-spinal cord barrier (BSCB) of a vertebral subject, particularly a human.

To this end, the implantable device can be used together with an ultrasound (US) contrast agent, which will be administered before or during the application, to the spinal cord of the subject, of the US beams. Alternatively, the implantable device can be used in absence of any US contrast agent.

The US contrast agent may be administered by injection, preferably by systemic injection. Systemic administration (e.g., intravenously) is a route of administration of an agent into the circulatory system so that the entire body is affected.

In some embodiments, the ultrasound contrast agent is injected into the bloodstream of the subject.

Preferably, the ultrasound contrast agent is administered as a bolus just before or just after the US beam application. Preferably, the US contrast agent is administered just after the US beam application. More preferably, the US contrast agent is administered between 0 and 10 seconds after the US beam application. Advantageously, the US contrast agent administration is performed during 15/30 seconds long. The US beams can last for several minutes, generally for 4 minutes. When successive US beams are applied, the ultrasound contrast agent is preferably delivered only once, just before the first US beam application, though it may be delivered by a continuous infusion through the activation of successive US beams.

According to the invention, the ultrasound contrast agent may contain gaseous bubbles, a high concentration of gas, solid particles configured to vaporize in response to ultrasound, liquid configured to vaporize in response to ultrasound, micro particles configured to act as cavitation sites, solid particles having higher acoustic impedance than tissue in the desired region, and/or liquid with a high acoustic absorption coefficient.

In some embodiments, the ultrasound contrast agent is a microbubble contrast agent, preferably selected from the group consisting of sulphur hexafluoride microbubbles (SonoVue®), microbubbles made of an albumin shell and octafluoropropane gas core (Optison®), perflexane microbubbles encapsulated in an outer lipid shell (Imagent®), microbubbles made of octafluoropropane gas core encapsulated in an outer lipid shell (Definity®), or perfluorobutaine and nitrogen gas encapsulated in a lipid shell (BR38—Schneider et al., 2011). Preferably, the ultrasound contrast agent consists of sulphur hexafluoride microbubbles.

The microbubbles may have a mean diameter in a range from 1 μm to 10 μm. In some embodiments, the microbubbles have a mean diameter in a range from 4 μm to 5 μm. In some other embodiments, the microbubbles have a mean diameter in a range from 2 to 6 μm.

In some embodiments, the dose of ultrasound contrast agent ranges between 0.2 and 0.4 ml/kg based on the total weight of the subject.

In some embodiments, a therapeutically active agent is used together with the ultrasound contrast agent. The therapeutically active agent is a drug that must be delivered to the spinal cord of the patient. The therapeutically active agent is administered by injection, preferably by systemic injection.

In a particular embodiment, the therapeutically active agent and the ultrasound contrast agent (and/or US beams) are administered sequentially. The ultrasound contrast agent and/or US beams may be administered within a suitable time window prior to the administration of the therapeutically active agent. For example, the ultrasound contrast agent is administered less than 1 hours prior to the administration of the therapeutically active agent. Preferably, the ultrasound contrast agent is administered 5-60 minutes (e.g., 10-60, 10-50, 10-40, 10-30, 10-20, 10-15, 30-40, 30-50, or 30-60 minutes) prior to the administration of the therapeutically active agent. In some embodiments, the ultrasound contrast agent and/or US beams is administered 10, 15, 20, 25, 30, 35, 40, 45 or 50 min prior to the administration of the therapeutically active agent. In one example, the ultrasound contrast agent is administered 10 minutes prior to the administration of the therapeutically active agent. Alternatively, the therapeutically active agent is administered prior to the administration of the ultrasound contrast agent and/or US beams.

Alternatively, the ultrasound contrast agent and the therapeutically active agent may be administered concomitantly, or simultaneously, (e.g., by way of a same solution).

The “therapeutically active agent”, as used herein include any drug medicament, antibodies, glycoproteins, dissolution compounds, genetic materials such as RNA and DNA, stem cells, proteins or peptides, liposomes, lipids, synthetic or natural polymers or polymeric conjugates, macromolecules, nanocarriers, encapsulated drug molecules, pharmaceutical formulations, any other substance capable of producing therapeutic actions, and any mixtures thereof. In a particular embodiment, the therapeutically active agent is selected from growth factors, antibodies, stem cells, nanoparticles and liposomes.

Generally speaking, the use of an ultrasound contrast agent administered by injection to a subject, with the application of US beam to the spinal cord of said subject, facilitates the delivery of many agent (endogenous or exogenous agent) across the BSCB.

The implantable device may be used in a method for facilitating delivery of an agent (e.g., an endogenous or exogenous agent) across the BSCB of a subject, comprising administering to a subject in need thereof ultrasound contrast agent concomitantly with application of US beam to the spinal cord of the subject by use of the implantable device of the invention.

The combined use of ultrasound contrast agent and US beam not only facilitates delivery of endogenous molecules (e.g., molecules that are naturally present in the blood stream of the subject) across the BSCB, but also allows delivery of exogenous molecules (e.g., therapeutically active agents that are administered to the patient with the aim to target the spinal cord), across the BSCB. Systemic administration of ultrasound contrast agent concomitantly with the application of US beam to the spinal cord of the subject temporarily increases the permeability of the BSCB to these agents, thereby enhancing the delivery of the agents the spinal cord.

Accordingly, provided herein are methods for enhancing treatment of a spinal cord disorder by the co-use of ultrasound contrast agent and US beam, and optionally therapeutically active agent, wherein the US beams are applied to the spinal cord by use of the implantable device of the invention.

A method of treating a subject suffering from a spinal cord disorder is also provided, which comprises: administering to the subject an ultrasound contrast agent concomitantly with the application of the US beam to the spinal cord of the subject by use of one or more implantable device of the invention. Such method may be combined with the administration, in sequence or concomitantly with the ultrasound contrast agent, of a therapeutically active agent suitable to treat or prevent a spinal cord disorder.

The invention may be used for treating any kind of spinal cord disorder that may be treated by delivery of a drug present in the blood stream.

1-13. (canceled)

14. An implantable ultrasound (US) generating device for implantation within the vertebral column of a vertebrate subject, wherein the implantable US generating device comprises at least one US generating transducer suitable for emitting US beam(s) with an oblique orientation with respect to a longitudinal axis of a vertebral column.

15. The implantable device according to claim 14, wherein the US generating transducer is suitable for emitting US beam(s) with an angle between 45° and 65° with respect to the longitudinal axis of a vertebral column.

16. The implantable device according to claim 14, wherein the implantable device has a general shape of the posterior part of a vertebra.

17. The implantable device according to claim 14, wherein the US generating transducer is a plane US transducer with an oblique orientation within the implantable device.

18. The implantable device according to claim 14, wherein the US generating transducer is a plane US transducer with a longitudinal orientation within the implantable device, and the implantable device further comprises at least one lens to deviate US beam(s) in an oblique direction with respect to the longitudinal axis of the vertebral column.

19. The implantable device according to claim 14, wherein the US generating transducer is adapted to generate US beams with a resonance frequency ranging from 0.3 and 3 MHz, from 0.5 and 2 MHz, from 1 and 1.5 MHz, or about 0.75 MHz.

20. The implantable device according to claim 14, wherein the US generating transducer is adapted to generate US beams with a pressure level ranging from 0.3 to 2 MPa.

21. The implantable device according to claim 14, wherein the US generating transducer is adapted to generate unfocused US beams.

22. The implantable device according to claim 14, wherein said implantable device comprises two or more US generating transducers suitable for emitting US beam(s) with an oblique orientation with respect to a longitudinal axis of a vertebral column.

23. The implantable device according to claim 22, wherein a first US generating transducer is suitable for emitting US beam(s) upward and a second US generating transducer is suitable for emitting US beam(s) downward.

24. The implantable device according to claim 22, wherein the US generating transducers can be activated sequentially.

25. The implantable device according to claim 14, wherein at least one US generating transducer comprises an array of several transducers.

26. The implantable device according to claim 14, wherein said implantable device comprises transdermal connection means to be connected to an electric supplier.

27. A method of treating a subject suffering from a spinal cord disorder comprising the steps of: administering to the subject an ultrasound (US) contrast agent and applying US beam to the spinal cord of the subject by use of one or more US generating device implanted in the vertebral column of the subject, said US generating device comprising at least one US generating transducer suitable for emitting US beam(s) with an oblique orientation with respect to a longitudinal axis of the vertebral column of the subject.

28. The method according to claim 27, wherein the ultrasound contrast agent is administered as a bolus just before the US beam application.

29. The method according to claim 27, wherein the ultrasound contrast agent is administered as a bolus just after the US beam application.

30. The method according to claim 27, wherein the ultrasound contrast agent is selected from the group consisting of sulphur hexafluoride microbubbles, microbubbles made of an albumin shell and octafluoropropane gas core, perflexane microbubbles encapsulated in an outer lipid shell, microbubbles made of octafluoropropane gas core encapsulated in an outer lipid shell, or perfluorobutaine and nitrogen gas encapsulated in a lipid shell.

31. The method according to claim 27, wherein a therapeutically active agent is administered to the subject.

32. The method according to claim 31, wherein the ultrasound contrast agent is administered prior to the therapeutically active agent.
